(19) United States
(12) Patent Application Publication
(10) Pub. No.: US 2011/0130426 A1
(43) Pub. Date: Jun. 2, 2011

(54) Novel Sulphonamide Derivatives as Glucocorticoid Receptor Modulators for the Treatment of Inflammatory Diseases

(75) Inventors: Håkan Bladh, Lund (SE); Krister Henriksson, Lund (SE); Vijaykumar Hulikal, Bangalore (IN); Matti Lepistö, Lund (SE)

(73) Assignee: ASTRAZENECA AB, Sodertalje (SE)

(21) Appl. No.: 11/718,203

(22) PCT Filed: Oct. 26, 2005
(86) PCT No.: PCT/SE05/01608

§ 371 Date: Feb. 27, 2008

(30) Foreign Application Priority Data
Oct. 29, 2004 (SE) 0402635-7

(51) Int. Cl. A61K 31/444; C07D 261/08; A61K 31/421; A61P 29/00; C07D 333/20; A61K 31/381; C07D 213/42; A61K 31/4402; C07D 409/12; A61K 31/4436; C07D 401/12; A61K 31/4439; C07D 409/14

(52) U.S. Cl. 514/333; 548/249; 514/374; 549/75; 514/438; 546/338; 514/357; 549/59; 514/444; 546/280.4; 514/336; 546/275.4; 514/341; 546/256

(57) ABSTRACT

A compound of formula (I) or a pharmaceutically acceptable salt thereof; compositions comprising them, processes for preparing them and their use in medical therapy (for example modulating the glucocorticoid receptor in a warm blooded animal).

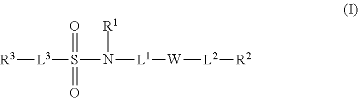

The present invention relates to sulphonamide derivatives, to pharmaceutical compositions comprising them, to processes for preparing them and to their use as medicaments (for example in the treatment of an inflammatory disease state).

Sulphonamide derivatives are disclosed as disinfectants in WO 2004018414. Sulphonamides are also disclosed in WO 99/38845.

It is known that certain non-steroidal compounds interact with the glucocorticoid receptor (GR) and, as a result of this interaction, produce a suppression of inflammation (see, for example, U.S. Pat. No. 6,323,199). Such compounds can show a clear dissociation between anti-inflammatory and metabolic actions making them superior to earlier reported steroidal and non-steroidal glucocorticoids. The present invention provides further non-steroidal compounds as modulators (for example agonists, antagonists, partial agonists or partial antagonists) of the glucocorticoid receptor capable of having a dissociation between their anti-inflammatory and metabolic actions.

The present invention provides a compound of formula (I):

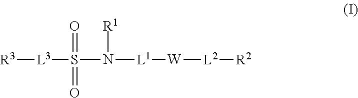

wherein:
R³ is phenyl optionally substituted by U, X, Y and Z; or thienyl, furyl or pyrazolyl all optionally substituted by X, Y and Z;
L³ is a bond or CH₂;
U, X, Y and Z are, independently, hydrogen, halo, C₁₋₆ alkyl, C₁₋₆ alkoxy, C₁₋₄ alkylthio, C₁₋₄ fluoroalkyl, C₁₋₄ fluoroalkoxy, phenyl (optionally substituted by halo, C₁₋₄ alkyl, C₁₋₄ fluoroalkyl, C₁₋₄ alkoxy or C₁₋₄ fluoroalkoxy), pyridyl (optionally substituted by halo, C₁₋₄ alkyl, C₁₋₄ fluoroalkyl, C₁₋₄ alkoxy or C₁₋₄ fluoroalkoxy), pyrazolyl (optionally substituted by halo, C₁₋₄ alkyl, C₁₋₄ fluoroalkyl, C₁₋₄ alkoxy or C₁₋₄ fluoroalkoxy), benzyloxy (optionally substituted by halo, C₁₋₄ alkyl, C₁₋₄ fluoroalkyl, C₁₋₄ alkoxy or C₁₋₄ fluoroalkoxy), phenoxy (optionally substituted by halo, C₁₋₄ alkyl, C₁₋₄ fluoroalkyl, C₁₋₄ alkoxy or C₁₋₄ fluoroalkoxy), pyridyloxy (optionally substituted by halo, C₁₋₄ alkyl, C₁₋₄ fluoroalkyl, C₁₋₄ alkoxy or C₁₋₄ fluoroalkoxy), nitro, cyano, S(O)₂NH₂, C(O)(C₁₋₄ alkyl), C(O)NH₂, NHC(O)(C₁₋₄ alkyl) or NR⁴R⁵; or X and Y join to form a fused benzene or pyridine ring;
R⁴ and R⁵ are, independently hydrogen, C₁₋₄ alkyl or C₃₋₇ cycloalkyl;
R¹ is hydrogen or C₁₋₄ alkyl;
W is a phenyl, isoxazolyl or pyrazolyl, cyclohexyl ring, or an acenaphthene ring system; W being optionally substituted by halo or C₁₋₄ alkyl;
L¹ is a bond or CH₂,
L² is a bond, O, NH, (CH₂)ₙ, or CH₂NH;
n is 1 or 2;
R² is cyclohexyl, phenyl, methylenedioxyphenyl, thienyl, pyrazolyl, thiazolyl, isoxazolyl, pyridinyl, pyrimidinyl, pyridazinyl, pyrazinyl, 1,3,5-triazinyl, 1,2,3-triazinyl, 1,2,4-triazinyl, benzofuranyl, benzthienyl, indolyl, indolinyl, dihydroindolinyl, indazolyl, benzimidazolyl, benzoxazolyl, benzthiazolyl, quinolinyl, tetrahydroquinolinyl, isoquinolinyl, quinoxalinyl, quinazolinyl, cinnolinyl, phthalazinyl, [1,8]-naphthiridinyl, [1,6]-naphthiridinyl, quinolin-2(1H)-onyl, isoquinolin-1(2H)-onyl, phthalazin-1(2H)-onyl, 1H-indazolyl, 1,3-dihydro-2H-indol-2-onyl, isoindolin-1-onyl, 3,4-dihydro-1H-isochromen-1-onyl, 1H-isochromen-1-onyl, or an acenaphthene ring system;
R² is optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR⁶R⁷;
R⁶ and R⁷ are, independently, hydrogen, C₁₋₄ alkyl or C₃₋₇ cycloalkyl;
or a pharmaceutically acceptable salt thereof.

Compounds of formula (I) can exist in different isomeric forms (such as enantiomers, diastereomers, geometric isomers or tautomers). The present invention covers all such isomers and mixtures thereof in all proportions.

Suitable salts include acid addition salts such as a hydrochloride, hydrobromide, phosphate, acetate, fumarate, maleate, tartrate, citrate, oxalate, methanesulphonate, p-toluenesulphonate, succinate, glutarate or malonate.

The compounds of formula (I) may exist as solvates (such as hydrates) and the present invention covers all such solvates.

Alkyl groups and moieties are straight or branched chain and are, for example, methyl, ethyl, n-propyl, iso-propyl, n-butyl, sec-butyl or tert-butyl.

Fluoroalkyl comprises, for example, 1 to 6, such as 1, 2, 3, 4 or 5 fluorine atoms. It is, for example, CHF₂, CF₃, CH₂CF₃ or C₂F₅. Fluoroalkoxy comprises, for example, 1 to 6, such as 1, 2, 3, 4 or 5 fluorine atoms. It is, for example, OCHF₂, OCF₃, OCH₂CF₃ or OC₂F₅.

Cycloalkyl is for example, cyclopropyl, cyclopentyl or cyclohexyl.

In one particular aspect the present invention provides a compound of formula (I) wherein:

R³ is phenyl optionally substituted by U, X, Y and Z;
L³ is a bond;
U, X, Y and Z are, independently, hydrogen, halo, C₁₋₆ alkyl, C₁₋₆ alkoxy, C₁₋₄ alkylthio, CF₃, OCF₃, phenyl (optionally substituted by halo or C₁₋₄ alkyl), benzyloxy, phenoxy (optionally substituted by halo or C₁₋₄ alkyl), nitro, cyano, S(O)₂NH₂, C(O)(C₁₋₄ alkyl), C(O)NH₂, NHC(O)(C₁₋₄ alkyl) or NR⁴R⁵; or X and Y join to form a fused benzene or pyridine ring;
R⁴ and R⁵ are, independently hydrogen, C₁₋₄ alkyl or C₃₋₇ cycloalkyl;
R¹ is hydrogen or C₁₋₄ alkyl;
W is a phenyl, isoxazolyl or pyrazolyl, cyclohexyl ring, or an acenaphthene ring system; W being optionally substituted by halo or C₁₋₄ alkyl;
L¹ is a bond or CH₂;
L² is a bond, O, NH, (CH₂)ₙ or CH₂NH;
n is 1 or 2;
R² is a phenyl, pyridyl or pyrazolyl ring, said ring being optionally substituted by halo, C₁₋₄ alkyl, C₁₋₄ alkoxy, C₁₋₄ alkylthio, CF₃, OCF₃, benzyloxy, nitro, cyano, S(O)₂NH₂, C(O)(C₁₋₄ alkyl), C(O)NH₂, NHC(O)(C₁₋₄ alkyl) or NR⁸R⁹;
R⁸ and R⁹ are, independently hydrogen, C₁₋₄ alkyl or C₃₋₇ cycloalkyl;
or a pharmaceutically acceptable salt thereof; for use as a medicament.

In another aspect the present invention provides a compound of formula (I) wherein:

R³ is phenyl optionally substituted by U, X, Y and Z; or thienyl, furyl or pyrazolyl all optionally substituted by X, Y and Z;
L³ is a bond or CH₂;
U, X, Y and Z are, independently, hydrogen, halo, C₁₋₆ alkyl, C₁₋₆ alkoxy, C₁₋₄ alkylthio, C₁₋₄ fluoroalkyl, C₁₋₄ fluoroalkoxy, phenyl (optionally substituted by halo, C₁₋₄ alkyl, C₁₋₄ fluoroalkyl, C₁₋₄ alkoxy or C₁₋₄ fluoroalkoxy), pyridyl (optionally substituted by halo, C₁₋₄ alkyl, C₁₋₄ fluoroalkyl, C₁₋₄ alkoxy or C₁₋₄ fluoroalkoxy), pyrazolyl (optionally substituted by halo, C₁₋₄ alkyl, C₁₋₄ fluoroalkyl, C₁₋₄ alkoxy or C₁₋₄ fluoroalkoxy), benzyloxy (optionally substituted by halo, C₁₋₄ alkyl, C₁₋₄ fluoroalkyl, C₁₋₄ alkoxy or C₁₋₄ fluoroalkoxy), phenoxy (optionally substituted by halo, C₁₋₄ alkyl, C₁₋₄ fluoroalkyl, C₁₋₄ alkoxy or C₁₋₄ fluoroalkoxy), pyridyloxy (optionally substituted by halo, C₁₋₄ alkyl, C₁₋₄ fluoroalkyl, C₁₋₄ alkoxy or C₁₋₄ fluoroalkoxy), nitro, cyano, S(O)₂NH₂, C(O)(C₁₋₄ alkyl), C(O)NH₂, NHC(O)(C₁₋₄ alkyl) or NR⁴R⁵; or X and Y join to form a fused benzene or pyridine ring;
R⁴ and R⁵ are, independently hydrogen, C₁₋₄ alkyl or C₃₋₇ cycloalkyl;
R¹ is hydrogen or C₁₋₄ alkyl;
W is cyclohexyl, phenyl, methylenedioxyphenyl, thienyl, pyrazolyl, thiazolyl, isoxazolyl, pyridinyl, pyrimidinyl, pyridazinyl, pyrazinyl, 1,3,5-triazinyl, 1,2,3-triazinyl, 1,2,4-triazinyl, benzofuranyl, benzthienyl, indolyl, indolinyl, dihydroindolinyl, indazolyl, benzimidazolyl, benzoxazolyl, benzthiazolyl, quinolinyl, tetrahydroquinolinyl, isoquinolinyl, quinoxalinyl, quinazolinyl, cinnolinyl, phthalazinyl, [1,8]-naphthiridinyl, [1,6]-naphthiridinyl, quinolin-2(1H)-onyl, isoquinolin-[(21])-onyl, phthalazin-1(2H)-onyl, 1H-indazolyl, 1,3-dihydro-2H-indol-2-onyl, isoindolin-1-onyl, 3,4-dihydro-1H-isochromen-1-only, 1H-isochromen-1-only, or an acenaphthene ring system;
W is optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, OH, C(O)₂H, C(O)₂(C₁₋₄ alkyl), S(O)₂(C₁₋₄), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR⁶R⁷;
R⁶ and R⁷ are, independently, hydrogen, C₁₋₄ alkyl or C₃₋₇ cycloalkyl;
L¹ is a bond or CH₂;
L² is a bond, O, NH, (CH₂)ₙ or CH₂NH;
n is 1 or 2;
R² is a phenyl, pyridyl or pyrazolyl ring, said ring being optionally substituted by halo, C₁₋₄ alkyl, C₁₋₄ alkoxy, C₁₋₄ alkylthio, CF₃, OCF₃, benzyloxy, nitro, cyano, S(O)₂NH₂, C(O)(C₁₋₄ alkyl), C(O)NH₂, NHC(O)(C₁₋₄ alkyl) or NR⁸R⁹;
R⁸ and R⁹ are, independently hydrogen, C₁₋₄ alkyl or C₃₋₇ cycloalkyl;
or a pharmaceutically acceptable salt thereof; provided that when L¹ and L³ are both a bond then W is not optionally substituted phenyl; and that when L¹ and L³ are both a bond, L² is (CH₂)₂, W is unsubstituted 2-pyridin-6-yl and R² is unsubstituted phenyl then R³ is neither 4-NHC(O)CH₃-phenyl nor —NH₂-phenyl.

In yet another aspect the present invention provides a compound of formula (I) wherein: R³ is phenyl optionally substituted by U, X, Y and Z; L³ is a bond; U, X, Y and Z are, independently, hydrogen, halo (such as fluoro or chloro), C₁₋₆ alkyl, C₁₋₆ alkoxy, phenyl (optionally substituted by halo) or phenoxy (optionally substituted by C₁₋₄ alkyl); or X and Y join to form a fused benzene ring; R¹ is hydrogen or C₁₋₄ alkyl; W is a phenyl, isoxazolyl or pyrazolyl, cyclohexyl ring, or an acenaphthene ring system; W being optionally substituted by C₁₋₄ alkyl; L¹ is a bond or CH₂; L² is a bond, O, NH, (CH₂)ₙ or CH₂NH; n is 1 or 2; R² is a phenyl, pyridyl or pyrazolyl ring, said ring being optionally substituted by halo (such as chloro or bromo), C₁₋₄ alkyl, C₁₋₄ alkoxy or C₁₋₄ alkylthio; or a pharmaceutically acceptable salt thereof; for use as a medicament.

In a further aspect the present invention provides a compound of formula (I) wherein R³ is phenyl optionally substituted by U, X, Y and Z; L³ is a bond; U, X, Y and Z are, independently, hydrogen, halo, C₁₋₆ alkyl, C₁₋₆ alkoxy, C₁₋₄ alkylthio, CF₃, OCF₃, phenyl (optionally substituted by halo or C₁₋₄ alkyl), benzyloxy, phenoxy (optionally substituted by halo or C₁₋₄ alkyl), nitro, cyano, S(O)₂NH₂, C(O)(C₁₋₄ alkyl), C(O)NH₂, NHC(O)(C₁₋₄ alkyl) or NR⁴R⁵; or X and Y join to form a fused benzene or pyridine ring; R⁴ and R⁵ are, independently hydrogen, C₁₋₄ alkyl or C₃₋₇ cycloalkyl; R¹ is hydrogen or C₁₋₄ alkyl; W is a phenyl, isoxazolyl or pyrazolyl, cyclohexyl ring, or an acenaphthene ring system; W being optionally substituted by halo or C₁₋₄ alkyl; L¹ is a bond or CH₂; L² is a bond, O, NH, (CH₂)ₙ or CH₂NH; n is 1 or 2; R² is a phenyl, pyridyl or pyrazolyl ring, said ring being optionally substituted by halo, C₁₋₄ alkyl, C₁₋₄ alkoxy, C₁₋₄ alkylthio, CF₃, OCF₃, benzyloxy, nitro, cyano, S(O)₂NH₂, C(O)(C₁₋₄ alkyl), C(O)NH₂, NHC(O)(C₁₋₄ alkyl) or NR⁸R⁹; R⁸ and R⁹ are, independently hydrogen, C₁₋₄ alkyl or C₃₋₇ cycloalkyl; or a pharmaceutically acceptable salt thereof.

In a still further aspect the present invention provides a compound of formula (I) wherein: R³ is phenyl optionally substituted by U, X, Y and Z; L³ is a bond; U, X, Y and Z are, independently, hydrogen, halo, C₁₋₆ alkyl, C₁₋₆ alkoxy, C₁₋₄ alkylthio, CF₃, OCF₃, phenyl (optionally substituted by halo or C₁₋₄ alkyl), benzyloxy, phenoxy (optionally substituted by halo or C₁₋₄ alkyl), nitro, cyano, S(O)₂NH₂, C(O)(C₁₋₄ alkyl), C(O)NH₂, NHC(O)(C₁₋₄ alkyl) or NR⁴R⁵; or X and Y join to form a fused benzene or pyridine ring; R⁴ and R⁵ are, independently hydrogen, C₁₋₄ alkyl or C₃₋₇ cycloalkyl; R¹ is hydrogen or C₁₋₄ alkyl; W is a phenyl or isoxazolyl, cyclohexyl ring, or an acenaphthene ring system; W being optionally substituted by halo or C₁₋₄ alkyl; L¹ is a bond or CH₂; L² is a bond, O, NH or (CH₂)ₙ; n is 1 or 2; R² is a phenyl, pyridyl or pyrazolyl ring, said ring being optionally substituted by halo, C₁₋₄ alkyl, C₁₋₄ alkoxy, C₁₋₄ alkylthio, CF₃, OCF₃, benzyloxy, nitro, cyano, S(O)₂NH₂, C(O)(C₁₋₄ alkyl), C(O)NH₂, NHC(O)(C₁₋₄ alkyl) or NR⁸R⁹; R⁸ and R⁹ are, independently hydrogen, C₁₋₄ alkyl or C₃₋₇ cycloalkyl; or a pharmaceutically acceptable salt thereof.

In another aspect the present invention provides a compound of formula (I) wherein L¹ is CH₂.

In yet another aspect the present invention provides a compound of formula (I) wherein L³ is a bond.

In a further aspect the present invention provides a compound of formula (I) wherein L² is CH₂CH₂.

In a still further aspect the present invention provides a compound of formula (I) wherein W is phenyl (for example unsubstituted phenyl).

In another aspect the present invention provides a compound of formula (I) wherein R³ is phenyl (optionally substituted by halogen, C₁₋₄ alkyl, C₁₋₄ haloalkyl, C₁₋₄ alkoxy or C₁₋₄ haloalkoxy), pyridyl (optionally substituted by halogen, C₁₋₄ alkyl, C₁₋₄ haloalkyl, C₁₋₄ alkoxy or C₁₋₄ haloalkoxy) or pyrazolyl (optionally substituted by C₁₋₄ alkyl, C₁₋₄ haloalkyl or phenyl (itself optionally substituted by halogen, C₁₋₄ alkyl, C₁₋₄ haloalkyl, C₁₋₄ alkoxy or C₁₋₄ haloalkoxy)).

In yet another aspect the present invention provides a compound of formula (I) wherein R³ is phenyl optionally substituted by halo, C₁₋₆ alkyl, C₁₋₆ alkoxy, C₁₋₄ alkylthio, CF₃, OCF₃, phenoxy (optionally substituted by halo or C₁₋₄ alkyl), phenyl (optionally substituted by halo or C₁₋₄ alkyl), nitro, cyano, S(O)₂NH₂, C(O)(C₁₋₄ alkyl), C(O)NH₂, NHC(O)(C₁₋₄ alkyl) or NR⁴R⁵; R⁴ and R⁵ are, independently, hydrogen, C₁₋₄ alkyl or C₃₋₇ cycloalkyl.

In a further aspect the present invention provides a compound of formula (I) wherein R³ is phenyl optionally substituted by halo, C₁₋₆ alkyl, C₁₋₆ alkoxy, C₁₋₄ alkylthio, CF₃, OCF₃, nitro, cyano, S(O)₂NH₂, C(O)(C₁₋₄ alkyl), C(O)NH₂, NHC(O)(C₁₋₄ alkyl) or NR⁴R⁵; R⁴ and R⁵ are, independently, hydrogen, C₁₋₄ alkyl or C₃₋₇ cycloalkyl.

In a still further aspect the present invention provides a compound of formula (I) wherein R² is phenyl, methylenedioxyphenyl, pyridinyl, benzofuranyl, benzthienyl, indolyl, indolinyl, dihydroindolinyl, benzimidazolyl, benzoxazolyl, benzthiazolyl, quinolinyl, indazolyl, tetrahydroquinolinyl, isoquinolinyl, quinoxalinyl, quinazolinyl, cinnolinyl, phthalazinyl, [1,8]-naphthiridinyl or [1,6]-naphthiridinyl, optionally substituted as defined above.

In another aspect the present invention provides a compound of formula (I) wherein R² is phenyl, pyridyl, indolyl (for example indol-4-yl, indol-5-yl, indol-6-yl or indol-7-yl), indazolyl (for example indazol-4-yl, indazol-5-yl, indazol-6-yl or indazol-7-yl), quinolinyl (for example quinolin-5-yl) or isoquinolinyl (for example isoquinolin-5-yl).

In yet another aspect the present invention provides a compound of formula (I) wherein R² is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃) or C(O)NH₂.

In a further aspect the present invention provides a compound of formula (I) wherein R² is a phenyl, pyridyl or pyrazolyl ring, said ring being optionally substituted by halo, C₁₋₄ alkyl, C₁₋₄ alkoxy, C₁₋₄ alkylthio, CF₃, OCF₃, benzyloxy, nitro, cyano, S(O)₂NH₂, C(O)(C₁₋₄ alkyl), C(O)NH₂, NHC(O)(C₁₋₄ alkyl) or NR⁸R⁹; and R⁸ and R⁹ are, independently hydrogen, C₁₋₄ alkyl or C₃₋₇ cycloalkyl.

In a still further aspect the present invention provides a compound of formula (I) wherein R¹ is hydrogen.

The compounds of the invention are illustrated by the Examples.

The compounds of formula (I) can be prepared using or adapting methods disclosed in the art, or by using or adapting the method disclosed in the Examples below. Starting materials for the preparative methods are either commercially available or can be prepared by literature methods, adapting literature methods.

For example the compounds of the invention can be prepared by coupling a compound of formula (II):

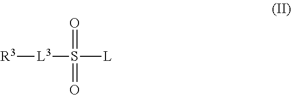

wherein L is a leaving group (for example chlorine), with a compound of formula (III):

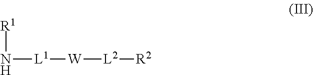

in a suitable solvent (such as tetrahydrofuran or N,N-dimethylformamide) at a temperature in the range −10° C. to 50° C.

The invention further provides processes for the preparation of these compounds of formula (I).

Because of their ability to bind to the glucocorticoid receptor the compounds of formula (I) are useful as anti-inflammatory agents, and can also display antiallergic, immunosuppressive and anti-proliferative actions. Thus, the compounds of formula (I) can be used as medicaments for treatment or prophylaxis of the following pathologic conditions in mammals (such as humans):
(i) Lung diseases, which coincide with inflammatory, allergic and/or proliferative processes:
   chronically obstructive lung diseases of any origin, mainly bronchial asthma
   bronchitis of different origins
   all forms of restructive lung diseases, mainly allergic alveolitis
   all forms of pulmonary edema, mainly toxic pulmonary edema
   sarcoidoses and granulomatoses, such as Boeck's disease
(ii) Rheumatic diseases/auto-immune diseases/degenerative joint diseases, which coincide with inflammatory, allergic and/or proliferative processes:
   all forms of rheumatic diseases, especially rheumatoid arthritis, acute rheumatic fever, polymyalgia rheumatica, collagenoses
   reactive arthritis
   inflammatory soft-tissue diseases of other origins
   arthritic symptoms in degenerative joint diseases (arthroses)
   traumatic arthritides
   collagen diseases of other origins, for example systemic lupus erythematodes, sclerodermia, polymyositis, dermatomyositis, polyarteritis nodosa, temporal arteritis
   Sjögren's syndrome, Still syndrome, Felty's syndrome
(iii) Allergies, which coincide with inflammatory, allergic and/or proliferative processes:
   All forms of allergic reactions, for example Quincke's edema, hay fever, insect bites, allergic reactions to pharmaceutical agents, blood derivatives, contrast media, etc., anaphylactic shock, urticaria, contact dermatitis
(iv) Dermatological diseases, which coincide with inflammatory, allergic and/or proliferative processes:
   atopic dermatitis (mainly in children)
   psoriasis
   erythematous diseases, triggered by different noxae, for example radiation, chemicals, burns, etc.
   acid burns
   bullous dermatoses
   diseases of the lichenoid group
   itching (for example of allergic origins)
   seborrheal eczema
   rosacea
   pemphigus vulgaris
   erythema exudativum multiforme
   erythema nodosum
   balanitis
   vulvitis
   inflammatory hair loss, such as alopecia areata
   cutaneous T-cell lymphoma
(v) Nephropathies, which coincide with inflammatory, allergic and/or proliferative processes:
   nephrotic syndrome
   all nephritides
(vi) Liver diseases, which coincide with inflammatory, allergic and/or proliferative processes:
   acute liver cell decomposition
   acute hepatitis of different origins, for example virally-, toxically- or pharmaceutical agent-induced
   chronically aggressive and/or chronically intermittent hepatitis
(vii) Gastrointestinal diseases, which coincide with inflammatory, allergic and/or proliferative processes:
   regional enteritis (Crohn's disease)
   ulcerative colitis
   gastroenteritis of other origins, for example native sprue
(viii) Proctological diseases, which coincide with inflammatory, allergic and/or proliferative processes:
   anal eczema
   fissures
   haemorrhoids
   idiopathic proctitis
(ix) Eve diseases, which coincide with inflammatory, allergic and/or proliferative processes:
   allergic keratitis, uvenitis iritis
   conjunctivitis
   blepharitis
   optic neuritis
   chorioiditis
   sympathetic ophthalmia
(x) Diseases of the ear-nose-throat area, which coincide with inflammatory, allergic and/or proliferative processes:
   allergic rhinitis, hay fever
   otitis externa, for example caused by contact dermatitis, infection, etc.
   otitis media
(xi) Neurological diseases, which coincide with inflammatory, allergic and/or proliferative processes:
   cerebral edema, mainly tumor-induced cerebral edema
   multiple sclerosis
   acute encephalomyelitis
   different forms of convulsions, for example infantile nodding spasms
(xii) Blood diseases, which coincide with inflammatory, allergic and/or proliferative processes:
   acquired haemolytic anemia
   idiopathic thrombocytopenia
(xiii) Tumor diseases, which coincide with inflammatory, allergic and/or proliferative processes:
   acute lymphatic leukaemia
   malignant lymphoma
   lymphogranulomatoses
   lymphosarcoma
   extensive metastases, mainly in breast and prostate cancers
(xiv) Endocrine diseases, which coincide with inflammatory, allergic and/or proliferative processes:
   endocrine orbitopathy
   thyrotoxic crisis
   de Quervain's thyroiditis
   Hashimoto's thyroiditis
   hyperthyroidism
(xv) Transplants, which coincide with inflammatory, allergic and/or proliferative processes;
(xvi) Severe shock conditions, which coincide with inflammatory, allergic and/or proliferative processes, for example anaphylactic shock
(xvii) Substitution therapy, which coincides with inflammatory, allergic and/or proliferative processes, with:
   innate primary suprarenal insufficiency, for example congenital adrenogenital syndrome
   acquired primary suprarenal insufficiency, for example Addison's disease, autoimmune adrenalitis, meta-infective, tumors, metastases, etc.
   innate secondary suprarenal insufficiency, for example congenital hypopituitarism
   acquired secondary suprarenal insufficiency, for example meta-infective, tumors, etc.
(xviii) Emesis, which coincides with inflammatory, allergic and/or proliferative processes:
   for example in combination with a 5-HT₃-antagonist in cytostatic-agent-induced vomiting.

Without prejudice to the foregoing, the compounds of formula (I) can also be used to treat disorders such as: Conies Syndrome, primary and secondary hyperaldosteronism, increased sodium retention, increased magnesium and potassium excretion (diuresis), increased water retention, hypertension (isolated systolic and combined systolic/diastolic), arrhythmias, myocardial fibrosis, myocardial infarction, Bartter's Syndrome, disorders associated with excess catecholamine levels, diastolic and systolic congestive heart failure (CHF), peripheral vascular disease, diabetic nephropathy, cirrhosis with edema and ascites, oesophageal varicies, Addison's Disease, muscle weakness, increased melanin pigmentation of the skin, weight loss, hypotension, hypoglycemia, Cushing's Syndrome, obesity, hypertension, glucose intolerance, hyperglycemia, diabetes mellitus, osteoporosis, polyuria, polydipsia, inflammation, autoimmune disorders, tissue rejection associated with organ transplant, malignancies such as leukemias and lymphomas, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hyperglycemia, acute adrenal insufficiency, chronic primary adrenal insufficiency, secondary adrenal insufficiency, congenital adrenal hyperplasia, cerebral edema, thrombocytopenia, and Little's syndrome, systemic inflammation, inflammatory bowel disease, systemic lupus erythematosus, discoid lupus erythematosus, polyartitis nodosa, Wegener's granulomatosis, giant cell arthritis, rheumatoid arthritis, osteoarthritis, hay fever, allergic rhinitis, contact dermatitis, atopic dermatitis, exfoliative dermatitis, urticaria, angioneurotic edema, chronic obstructive pulmonary disease, asthma, tendonitis, bursitis, Crohn's disease, ulcerative colitis, autoimmune chronic active hepatitis, hepatitis, cinhosis, inflammatory scalp alopecia, panniculitis, psoriasis, inflamed cysts, pyoderma gangrenosum, pemphigus vulgaris, bullous pemphigoid, dermatomyositis, eosinophilic fasciitis, relapsing polychondritis, inflammatory vasculitis, sarcoidosis Sweet's disease, type 1 reactive leprosy, capillary hemangiomas, lichen planus, erythema nodosum acne, hirsutism, toxic epidermal necrolysis, erythema multiform, cutaneous T-cell lymphoma, psychoses, cognitive disorders (such as memory disturbances) mood disorders (such as depression and bipolar disorder), anxiety disorders and personality disorders.

As used herein the term “congestive heart failure” (CHF) or ‘congestive heart disease” refers to a disease state of the cardiovascular system whereby the heart is unable to efficiently pump an adequate volume of blood to meet the requirements of the body's tissues and organ systems. Typically, CHF is characterized by left ventricular failure (systolic dysfunction) and fluid accumulation in the lungs, with the underlying cause being attributed to one or more heart or cardiovascular disease states including coronary artery disease, myocardial infarction, hypertension, diabetes, valvular heart disease, and cardiomyopathy. The term “diastolic congestive heart failure” refers to a state of CHF characterized by impairment in the ability of the heart to properly relax and fill with blood. Conversely, the term “systolic congestive heart failure” refers to a state of CHF characterized by impairment in the ability of the heart to properly contract and eject blood.

As will be appreciated by one of skill in the art, physiological disorders may present as a “chronic” condition, or an “acute” episode. The term “chronic”, as used herein, means a condition of slow progress and long continuance. As such, a chronic condition is treated when it is diagnosed and treatment continued throughout the course of the disease. Conversely, the term “acute” means an exacerbated event or attack, of short course, followed by a period of remission. Thus, the treatment of physiological disorders contemplates both acute events and chronic conditions. In an acute event, compound is administered at the onset of symptoms and discontinued when the symptoms disappear.

In another aspect the present invention provides a compound or formula (I) or a pharmaceutically acceptable salt thereof, in the manufacture of a medicament for use in therapy (such as a therapy described above).

The present invention further provides a method of treating a glucocorticoid receptor mediated disease state in a mammal (such as man), which comprises administering to a mammal in need of such treatment an effective amount of a compound of formula (I), or a pharmaceutically acceptable salt thereof.

In a still further aspect of the invention a compound of formula (I) or a pharmaceutically acceptable salt thereof, is used to treat an inflammatory (such as an arthritic) condition.

In a still further aspect of the invention a compound of formula (I) or a pharmaceutically acceptable salt thereof, is used to treat an asthmatic or dermatological condition.

In order to use a compound of formula (I), or a pharmaceutically acceptable salt thereof, for the therapeutic treatment of a mammal, said active ingredient is normally formulated in accordance with standard pharmaceutical practice as a pharmaceutical composition.

Therefore in another aspect the present invention provides a pharmaceutical composition comprising a compound of formula (I), or a pharmaceutically acceptable salt thereof (active ingredient), and a pharmaceutically acceptable adjuvant, diluent or carrier. In a further aspect the present invention provides a process for the preparation of said composition comprising mixing the active ingredient with a pharmaceutically acceptable adjuvant, diluent or carrier. Depending on the mode of administration, the pharmaceutical composition can comprise from 0.05 to 99% w (percent by weight), for example from 0.05 to 80% w, such as from 0.10 to 70% w (for example from 0.10 to 50% w), of active ingredient, all percentages by weight being based on total composition.

A pharmaceutical composition of the present invention may be administered in standard manner for the disease condition that it is desired to treat, for example by topical (such as to the lung and/or airways or to the skin), oral, rectal or parenteral administration. Thus, a the compound of formula (I), or a pharmaceutically acceptable salt thereof, may be formulated into the form of, for example, an aerosol, a powder (for example dry or dispersible), a tablet, a capsule, a syrup, a granule, an aqueous or oily solution or suspension, an (lipid) emulsion, a suppository, an ointment, a cream, drops, or a sterile injectable aqueous or oily solution or suspension.

A suitable pharmaceutical composition of this invention is one suitable for oral administration in unit dosage form, for example a tablet or capsule containing between 0.1 mg and 1 g of active ingredient.

In another aspect a pharmaceutical composition of the invention is one suitable for intravenous, subcutaneous or intramuscular injection.

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. Tablets may be enteric coated by conventional means, for example to provide a coating of cellulose acetate phthalate.

The invention further relates to combination therapies or compositions wherein a compound of formula (I), or a pharmaceutically acceptable salt thereof, or a pharmaceutical composition comprising a compound of formula (I), or a pharmaceutically acceptable salt thereof, is administered concurrently (possibly in the same composition) or sequentially with an agent for the treatment of any one of the above disease states.

In particular, for the treatment of the inflammatory diseases (for example rheumatoid arthritis, COPD, asthma or allergic rhinitis) a compound of the invention can be combined with a TNF-α inhibitor (such as an anti-TNF monoclonal antibody (such as Remicade, CDP-870 and D.sub2.E.sub7.), or a TNF receptor immunoglobulin molecule (such as Enbrel.reg.)), a non-selective COX-1/COX-2 inhibitor (such as piroxicam or diclofenac; a propionic acid such as naproxen, flubiprofen, fenoprofen, ketoprofen or ibuprofen; a fenamate such as mefenamic acid, indomethacin, sulindac or apazone; a pyrazolone such as phenylbutazone; or a salicylate such as aspirin), a COX-2 inhibitor (such as meloxicam, celecoxib, rofecoxib, valdecoxib or etoricoxib) low dose methotrexate, lefunomide; ciclesonide; hydroxychloroquine, d-penicillamine or auranofin, or parenteral or oral gold.

The present invention still further relates to the combination of a compound of the invention together with:
a leukotriene biosynthesis inhibitor, a 5-lipoxygenase (5-LO) inhibitor or a 5-lipoxygenase activating protein (FLAP) antagonist, such as zileuton, ABT-761, fenleuton, tepoxalin, Abbott-79175, Abbott-85761, an N-(5-substituted)-thiophene-2-alkylsulfonamide, a 2,6-di-tert-butylphenol hydrazones, a methoxytetrahydropyran such as Zeneca ZD-2138, SB-210661, a pyridinyl-substituted 2-cyanonaphthalene compound such as L-739,010; a 2-cyanoquinoline compound such as L-746,530; an indole or quinoline compound such as MK-591, MK-886 or BAY x 1005;
a receptor antagonist for a leukotriene LTB.sub4., LTC.sub4., LTD.sub4. or LTE.sub4. selected from the group consisting of a phenothiazin-3-one such as L-651,392; an amidino compound such as CGS-25019c; a benzoxalamine such as ontazolast; a benzenecarboximidamide such as BILL 284/260; or a compound such as zafirlukast, ablukast, montelukast, pranlukast, verlukast (MK-679), RG-12525, Ro-245913, iralukast (CGP 45715A) or BAY x 7195;
a PDE4 inhibitor including an inhibitor of the isoform PDE4D;
an antihistaminic H.sub1. receptor antagonist such as cetirizine, loratadine, desloratadine, fexofenadine, astemizole, azelastine or chlorpheniramine;
a gastroprotective H.sub2. receptor antagonist;
an α.sub1.- and α.sub2.-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride or ethylnorepinephrine hydrochloride;
an anticholinergic agent such as ipratropium bromide, tiotropium bromide, oxitropium bromide, pirenzepine or telenzepine;
a β.sub1.- to β.sub4.-adrenoceptor agonist (such as β2 adrenoceptor agonist) such as metaproterenol, isoproterenol, isoprenaline, albuterol, salbutamol, formoterol, salmeterol, terbutaline, orciprenaline, bitolterol mesylate or pirbuterol, or a methylxanthanine including theophylline and aminophylline; sodium cromoglycate; or a muscarinic receptor (M1, M2, and M3) antagonist;
an insulin-like growth factor type I (IGF-1) mimetic;
an inhaled glucocorticoid with reduced systemic side effects, such as prednisone, prednisolone, flunisolide, triamcinolone acetonide, beclomethasone dipropionate, budesonide, fluticasone propionate or mometasone furoate;
an inhibitor of a matrix metalloprotease (MMP), such as a stromelysin, a collagenase, or a gelatinase or aggrecanase; such as collagenase-1 (MMP-1), collagenase-2 (MMP-8), collagenase-3 (MMP-13), stromelysin-1 (MMP-3), stromelysin-2 (MMP-10), and stromelysin-3 (MMP-11) or MMP-12;
a modulator of chemokine receptor function such as CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family;
an osteoporosis agent such as roloxifene, droloxifene, lasofoxifene or fosomax;
an immunosuppressant agent such as FK-506, rapamycin, cyclosporine, azathioprine or methotrexate;
a compound useful in the treatment of AIDS and/or HIV infection for example: an agent which prevents or inhibits the viral protein gp120 from engaging host cell CD4 {such as soluble CD4 (recombinant); an anti-CD4 antibody (or modified/recombinant antibody) for example PRO542; an anti-group120 antibody (or modified/recombinant antibody); or another agent which interferes with the binding of group120 to CD4 for example BMS806}; an agent which prevents binding to a chemokine receptor, other than CCR5, used by the HIV virus {such as a CXCR4 agonist or antagonist or an anti-CXCR4 antibody}; a compound which interferes in the fusion between the HIV viral envelope and a cell membrane {such as an anti-group 41 antibody; enfuvirtide (T-20) or T-1249}; an inhibitor of DC-SIGN (also known as CD209) {such as an anti-DC-SIGN antibody or an inhibitor of DC-SIGN binding}; a nucleoside/nucleotide analogue reverse transciptase inhibitor {for example zidovudine (AZT), nevirapine, didanosine (ddI), zalcitabine (ddC), stavudine (d4T), lamivudine (3TC), abacavir, adefovir or tenofovir (for example as free base or as disoproxil fumarate)}; a non-nucleoside reverse transciptase inhibitor {for example nevirapine, delavirdine or efavirenz}; a protease inhibitor {for example ritonavir, indinavir, saquinavir (for example as free base or as mesylate salt), nelfinavir (for example as free base or as mesylate salt), amprenavir, lopinavir or atazanavir (for example as free base or as sulphate salt)}; a ribonucleotide reductase inhibitor {for example hydroxyurea}; or an antiretroviral {for example emtricitabine}; or,
an existing therapeutic agent for the treatment of osteoarthritis, for example a non-steroidal anti-inflammatory agent (hereinafter NSAID's) such as piroxicam or diclofenac, a propionic acid such as naproxen, flubiprofen, fenoprofen, ketoprofen or ibuprofen, a fenamate such as mefenamic acid, indomethacin, sulindac or apazone, a pyrazolone such as phenylbutazone, a salicylate such as aspirin, a COX-2 inhibitor such as celecoxib, valdecoxib, rofecoxib or etoricoxib, an analgesic or intra-articular therapy such as a corticosteroid or a hyaluronic acid such as hyalgan or synvisc, or a P2X7 receptor antagonist.

The present invention still further relates to the combination of a compound of the invention together with: (i) a tryptase inhibitor; (ii) a platelet activating factor (PAF) antagonist; (iii) an interleukin converting enzyme (ICE) inhibitor; (iv) an IMPDH inhibitor;

(v) an adhesion molecule inhibitor including a VLA-4 antagonist; (vi) a cathepsin; (vii) a MAP kinase inhibitor; (viii) a glucose-6 phosphate dehydrogenase inhibitor; (ix) a kinin-B.sub1.- and B.sub2.-receptor antagonist; (x) an anti-gout agent, e.g., colchicine; (xi) a xanthine oxidase inhibitor, e.g., allopurinol; (xii) an uricosuric agent, e.g., probenecid, sulfinpyrazone or benzbromarone; (xiii) a growth hormone secretagogue; (xiv) a transforming growth factor (TGFβ); (xv) a platelet-derived growth factor (PDGF); (xvi) a fibroblast growth factor, e.g., basic fibroblast growth factor (bFGF); (xvii) a granulocyte macrophage colony stimulating factor (GM-CSF); (xviii) a capsaicin cream; (xix) a Tachykinin NK.sub1. and NK.sub3. receptor antagonist selected from the group consisting of NKP-608C; SB-233412 (talnetant); and D-4418; (xx) an elastase inhibitors selected from the group consisting of UT-77 and ZD-0892; (xxi) a TNFα converting enzyme inhibitor (TACE); (xxii) an induced nitric oxide synthase inhibitor (iNOS); or (xxiii) a chemoattractant receptor-homologous molecule expressed on TH2 cells (a CRTH2 antagonist).

The following compounds illustrate compounds of formula (I)

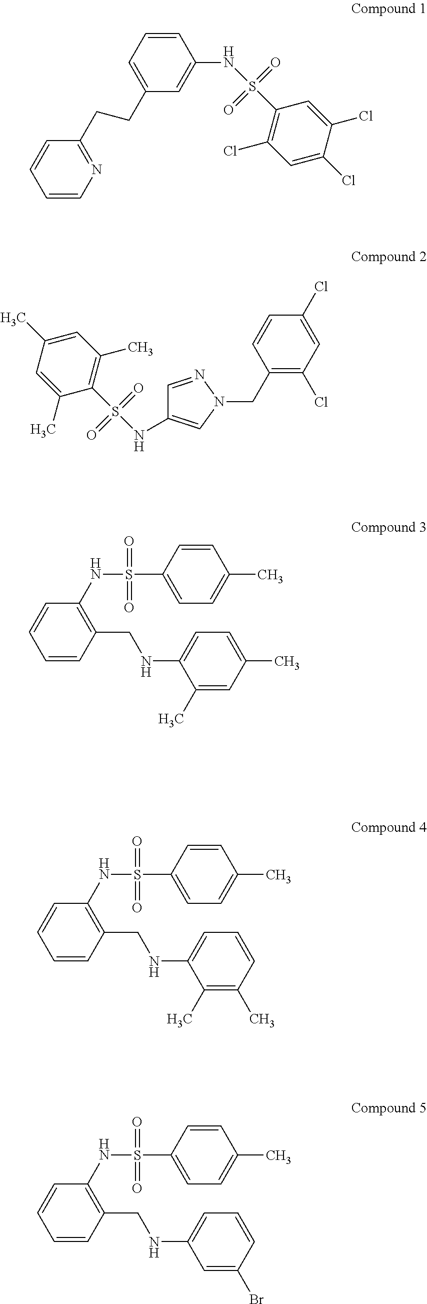

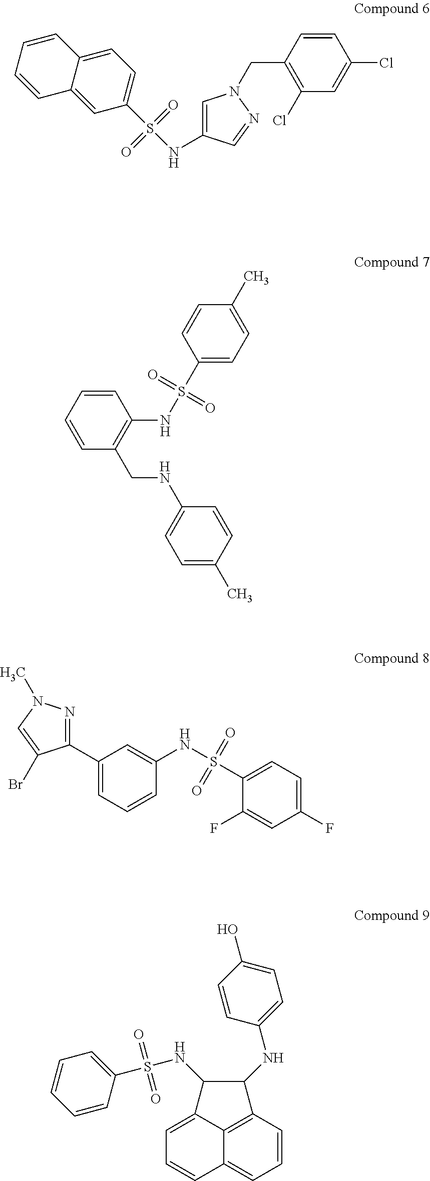

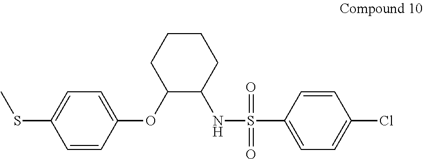

The following Examples illustrate the preparation of compounds of formula (I). In the Examples the following abbreviations are used

THF tetrahydrofuran
TFA trifluoroacetic acid
rt room temperature

### General Methods

¹H NMR spectra were recorded on a Varian Unity INOVA 400 MHz instrument. The central peaks of DMSO-d6 (δ_{H} 2.50 ppm) were used as internal references. Low resolution mass spectra and accurate mass determination were recorded on a Hewlett-Packard 1100 LC-MS system equipped with APCI ionisation chamber. Unless stated otherwise, starting materials were commercially available. All solvents and commercial reagents were of laboratory grade and were used as received.

The following method was used for LC/MS analysis:

Instrument Agilent 1100; Column C₁₈ Waters Symmetry 2.1×30 mm 3.5 μm; Flow rate 0.7 ml/min; Mass APCI; UV-absorption was measured at 254 nm; Solvent A: water+0.1% TFA; Solvent B: acetonitrile+0.1% TFA; Gradient 5-95%/B 8 min, 95% B 2 min.

### EXAMPLE 1

### 4-Methoxy-2,3,6-trimethyl-N-(5-methyl-3-phenyl-isoxazol-4-ylmethyl)-benzenesulfonamide

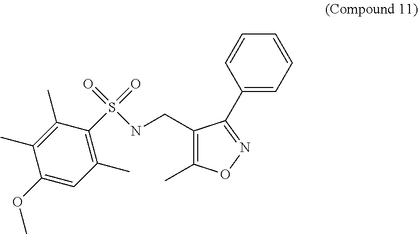

4-Methoxy-2,3,6-trimethyl-benzenesulfonyl chloride (120 μL 0.3M/THF) was mixed with [(5-methyl-3-phenylisoxazol-4-yl)methyl]amine (100 μL 0.3M/pyridine) and stirred overnight in ambient temperature before it was evaporated to dryness under reduced pressure. The residue was purified on HPLC-C₁₈ yielding 2.3 mg (20%)

APCI-MS m/z: 401.2 [MH+].

LC rt=5.8 min. UV 254 nm.

Examples 2-37 were synthesised by a method analogous to that described in Example 1 using the corresponding starting materials.

### EXAMPLE 2

### 4′-Fluoro-biphenyl-4-sulfonic acid (5-methyl-3-phenyl-isoxazol-4-ylmethyl)-amide

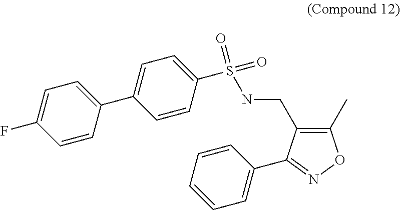

APCI-MS m/z: 423.2 [MH+].

LC rt=6.1 min. UV 254 nm.

### EXAMPLE 3

### 4-(1,1-Dimethyl-propyl)-N-(5-methyl-3-phenyl-isoxazol-4-ylmethyl)-benzenesulfonamide

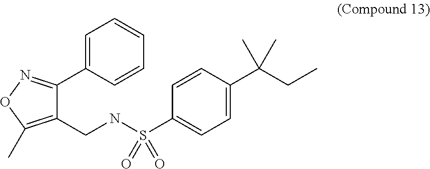

APCI-MS m/z: 399.2 [MH+].

LC rt=6.7 min. LTV 254 nm.

### EXAMPLE 4

### Naphthalene-2-sulfonic acid (5-methyl-3-phenyl-isoxazol-4-ylmethyl)-amide

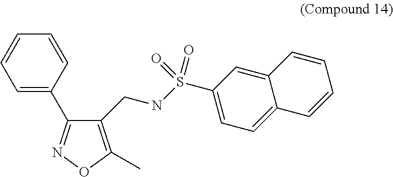

APCI-MS m/z: 379.1 [MH+].

LC rt=5.6 min. UV 254 nm.

### EXAMPLE 5

### Biphenyl-4-sulfonic acid (5-methyl-3-phenyl-isoxazol-4-ylmethyl)-amide

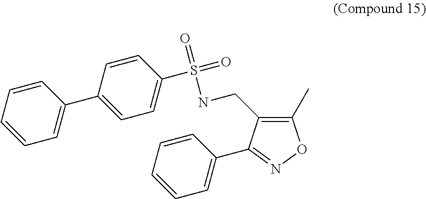

APCI-MS m/z: 405.2 [MH+].

LC rt=6.0 min. UV 254 nm.

### EXAMPLE 6

### 4-n-Butoxy-N-(5-methyl-3-phenyl-isoxazol-4-ylmethyl)-benzenesulfonamide

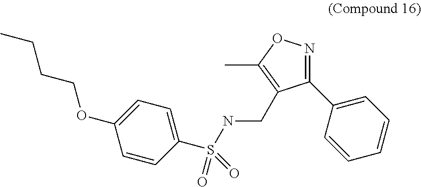

APCI-MS m/z: 401.2 [MH+].

LC rt=6.2 min. UV 254 nm.

### EXAMPLE 7

### 2,4,6-Trimethyl-N-(5-methyl-3-phenyl-isoxazol-4-ylmethyl)-benzenesulfonamide

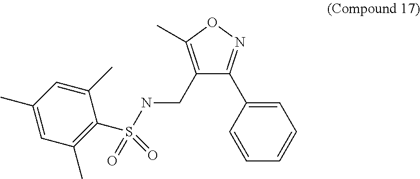

APCI-MS m/z: 371.2 [MH+].

LC rt=5.9 min. UV 254 nm.

### EXAMPLE 8

### N-(5-Methyl-3-phenyl-isoxazol-4-ylmethyl)-3-p-tolyloxy-benzenesulfonamide

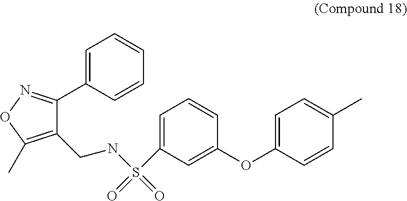

APCI-MS m/z: 435.2 [MH+].

LC rt=6.4 min. UV 254 nm.

### EXAMPLE 9

### N-{[b 5-(4-Chlorophenyl)-2-thienyl]methyl}-4-(trifluoromethoxy)benzenesulfonamide

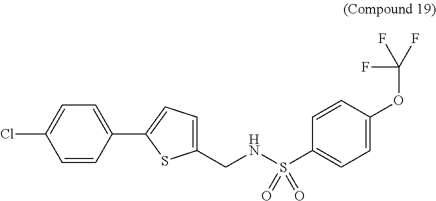

¹H NMR (399.99 MHz, DMSO) δ 8.50 (t, J=6.1 Hz, 1H), 7.91 (d, J=8.8 Hz, 2H), 7.56 (dd, J=8.2, 6.2 Hz, 4H), 7.44 (d, J=8.6 Hz, 2H), 7.28 (d, J=3.6 Hz, 1H), 6.89 (d, J=3.5 Hz, 1H), 4.25 (d, J=5.6 Hz, 2H) LC rt=6.9 min. UV 254 nm.

### EXAMPLE 10

### N-{[5-(4-Chlorophenyl)-2-thienyl]methyl}-4-(trifluoromethyl)benzenesulfonamide

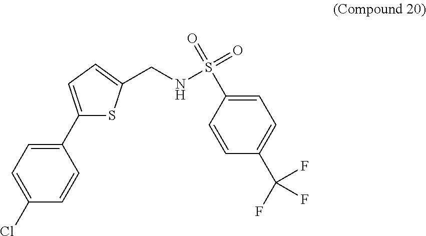

LC rt=6.8 min. UV 254 nm.

### EXAMPLE 11

### 2,4,6-Trimethyl-N-[3-(2-pyridin-2-ylethyl)phenyl]benzenesulfonamide

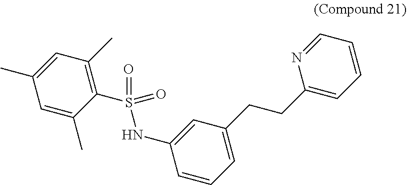

APCI-MS m/z: 381.2 [MH+].

LC rt 4.2 min. UV 254 nm.

### EXAMPLE 12

### 4-Bromo-N-{[5-(4-chlorophenyl)-2-thienyl]methyl}benzenesulfonamide

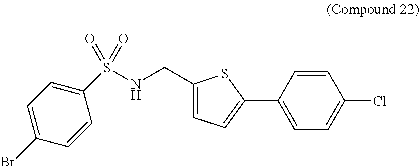

LC rt 6.7 min. UV 254 nm.

### EXAMPLE 13

### N-{[5-(4-chlorophenyl)-2-thienyl]methyl}-4-propylbenzenesulfonamide

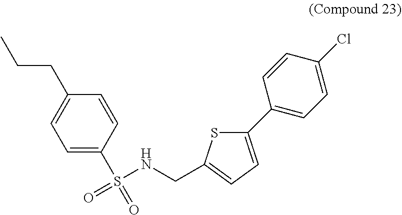

LC rt 7.1 min. UV 254 nm.

### EXAMPLE 14

### 4-Chloro-N-{[5-(4-chlorophenyl)-2-thienyl]methyl}benzenesulfonamide

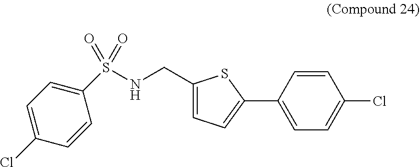

LC rt 6.6 min. UV 254 nm.

### EXAMPLE 15

### 5-Bromo-N-{[5-(4-chlorophenyl)-2-thienyl]methyl}thiophene-2-sulfonamide

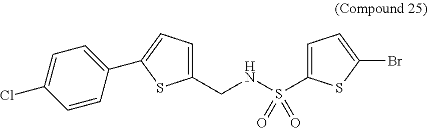

LC rt 6.7 min. UV 254 mm

### EXAMPLE 16

### 2,5-Dichloro-N-[3-(2-pyridin-2-ylethyl)phenyl]thiophene-3-sulfonamide

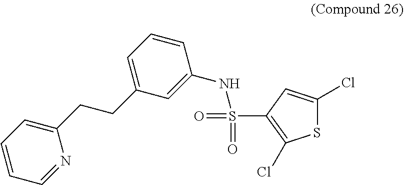

APCI-MS m/z: 413.0, 415.0 [MH+].

LC rt 4.1 min. LTV 254 nm.

### EXAMPLE 17

### N-{[5-(4-chlorophenyl)-2-thienyl]methyl}-3-(trifluoromethyl)benzenesulfonamide

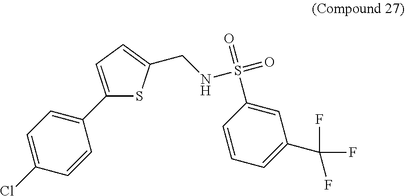

LC rt 6.7 min. UV 254 mm

### EXAMPLE 18

### 4-Bromo-N-{[5-(4-chlorophenyl)-2-thienyl]methyl}-2-methylbenzenesulfonamide

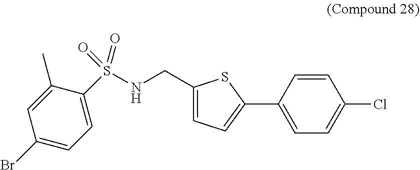

LC rt 7.0 min. UV 254 nm.

### EXAMPLE 19

### N-{[5-(4-Chlorophenyl)-2-thienyl]methyl}-2,4,6-trimethylbenzenesulfonamide

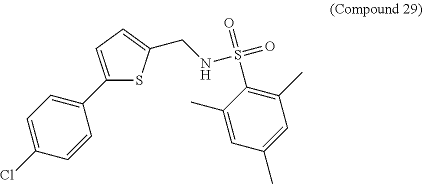

LC rt 7.1 min. UV 254 nm.

### EXAMPLE 20

### N-{[5-(4-Chlorophenyl)-2-thienyl]methyl}-4-nitrobenzenesulfonamide

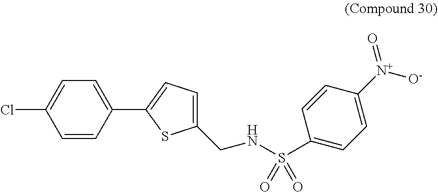

LC rt 6.2 min. UV 254 nm.

### EXAMPLE 21

### N-{[5-(4-Chlorophenyl)-2-thienyl]methyl}-4-fluorobenzenesulfonamide

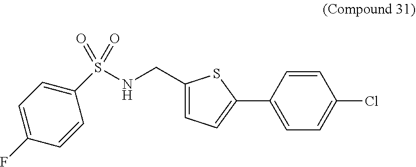

LC rt 6.3 min. UV 254 nm.

### EXAMPLE 22

### 5-Methyl-1-phenyl-N-[3-(2-pyridin-2-ylethyl)phenyl]-1H-pyrazole-4-sulfonamide

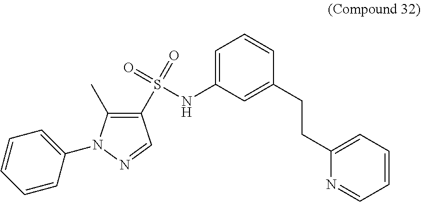

APCI-MS m/z: 419.2 [MH+].

LC rt 3.8 min. UV 254 nm

### EXAMPLE 23

### 5-[1-Methyl-5-(trifluoromethyl)-1H-pyrazol-3-yl]-N-[3-(2-pyridin-2-ylethyl)phenyl]-thiophene-2-sulfonamide

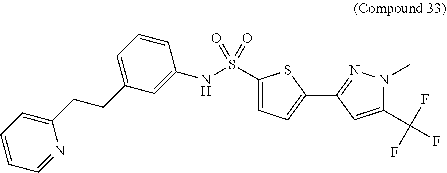

APCI-MS m/z: 493.1, 415.0 [MH+].

LC rt 4.6 min. UV 254 nm

### EXAMPLE 24

### 2-Bromo-N-{[5-(4-chlorophenyl)-2-thienyl]methyl}benzenesulfonamide

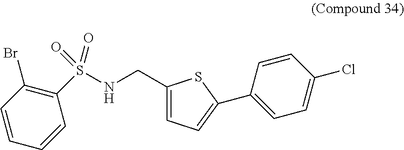

LC rt 6.5 min. UV 254 nm

### EXAMPLE 25

### N-{[5-(4-Chlorophenyl)-2-thienyl]methyl}-4-(pyridin-2-yloxy)benzenesulfonamide

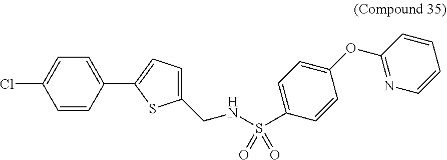

APCI-MS m/z: 457.1 [MH+].

LC rt 6.4 min. UV 254 nm

### EXAMPLE 26

### N-{[5-(4-Chlorophenyl)-2-thienyl]methyl}-4-methoxybenzenesulfonamide

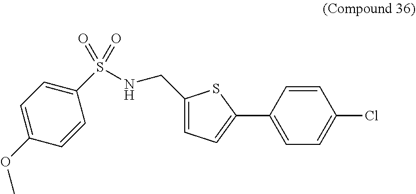

LC rt 6.2 min. UV 254 nm

### EXAMPLE 27

### 5-Pyridin-2-yl-N-[3-(2-pyridin-2-ylethyl)phenyl]thiophene-2-sulfonamide

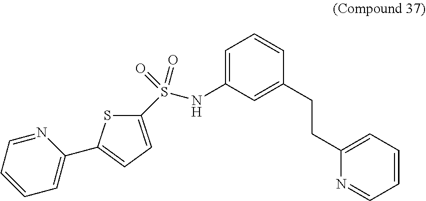

APCI-MS ink 422.2 [MH+].

LC rt 3.8 min. UV 254 nm

### EXAMPLE 28

### 2,5-dimethyl-N-[3-(2-pyridin-2-ylethyl)phenyl]thiophene-3-sulfonamide

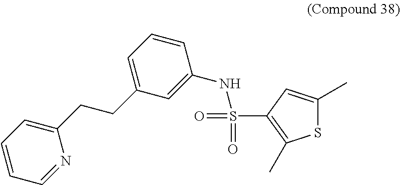

APCI-MS m/z: 373.1 [MH+].

LC rt 3.9 min. UV 254 nm

### EXAMPLE 29

### 3,5-Dimethyl-N-[3-(2-pyridin-2-ylethyl)phenyl]benzenesulfonamide

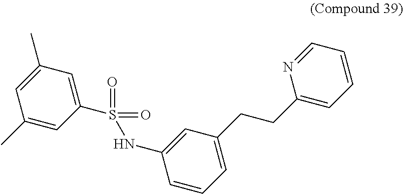

APCI-MS ink: 367.1 [MH+].

LC rt 4.0 min. UV 254 nm

### EXAMPLE 30

### N-{[5-(4-Chlorophenyl)-2-thienyl]methyl}-3-methoxybenzenesulfonamide

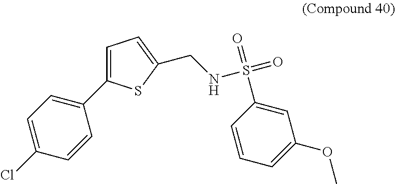

LC rt 6.3 min. UV 254 nm

### EXAMPLE 31

### N-{[5-(4-Chlorophenyl)-2-thienyl]methyl}-4-methoxy-2,3,6-trimethylbenzenesulfonamide

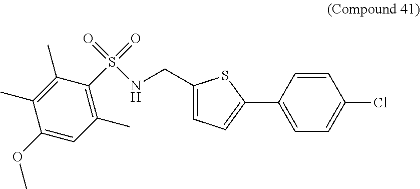

LC rt 7.0 min. UV 254 nm

### EXAMPLE 32

### N-{[5-(4-Chlorophenyl)-2-thienyl]methyl}-3-(4-methylphenoxy)benzenesulfonamide

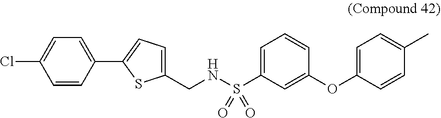

LC rt 7.3 min. LTV 254 nm

### EXAMPLE 33

### 5-Chloro-N-[3-(2-pyridin-2-ylethyl)phenyl]thiophene-2-sulfonamide

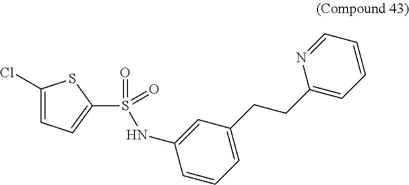

APCI-MS m/z 379.0 [MH+].

LC rt 3.9 min. LTV 254 nm

### EXAMPLE 34

### 1-(4-Chlorophenyl)-N-{[5-(4-chlorophenyl)-2-thienyl]methyl}methanesulfonamide

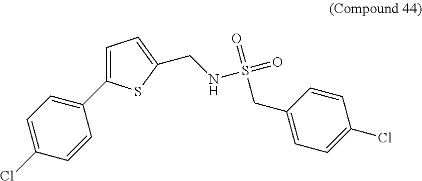

LC rt 6.6 min. UV 254 nm

### EXAMPLE 35

### 2,5-Dimethyl-N-[3-(2-pyridin-2-ylethyl)phenyl]furan-3-sulfonamide

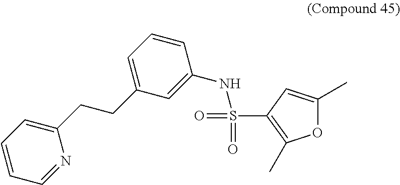

APCI-MS m/z: 357.1 [MH+].

LC rt 3.7 min. UV 254 nm

### EXAMPLE 36

### 1-(Difluoromethyl)-3,5-dimethyl-N-[3-(2-pyridin-2-ylethyl)phenyl]-1H-pyrazole-4-sulfonamide

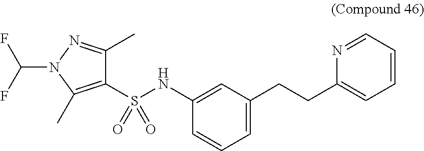

APCI-MS m/z: 407.2 [MH+].

LC rt 3.6 min. UV 254 nm

### EXAMPLE 37

### N-{[5-(4-Chlorophenyl)-2-thienyl]methyl}-3-cyanobenzenesulfonamide

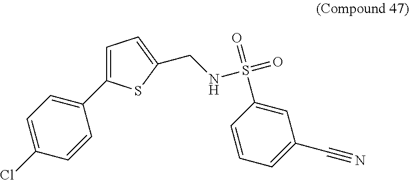

LC rt 6.1 min. UV 254 nm

### EXAMPLE 38

### 2,4-Dimethyl-N-[3-(2-pyridin-2-ylethyl)phenyl]benzenesulfonamide

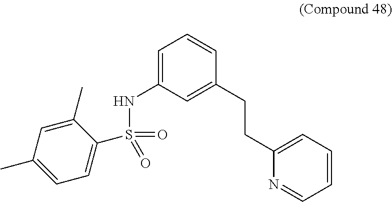

Step 1: 2,4-Dimethylbenzenesulfonyl chloride

2,4-dimethylbenzenesulfonic acid (10 mmole, 1.86 g), DIEA (10 mmole, 1.7 mL) and cyanuric chloride (10 mmole, 1.84 g) were dissolved in acetone (40 mL) and the reaction mixture was refluxed overnight. After cooling to room temperature the mixture was filtered through a Celite pad. Solvent was removed by evaporation under reduced pressure. The product was used in the next step without any further purification.

Step 2: 2,4-Dimethyl-N-[3-(2-pyridin-2-ylethyl)phenyl]benzenesulfonamide

Was synthesised by a method analogous to that described in Example 1 using the corresponding starting materials.

APCI-MS m/z: 367.1 [MH+].

LC rt 4.0 min. UV 254 nm

Example 39-40 were synthesised by a method analogous to that described in Example 38 using the corresponding starting materials.

### EXAMPLE 39

### 2,5-Dimethyl-N-[3-(2-pyridin-2-ylethyl)phenyl]benzenesulfonamide

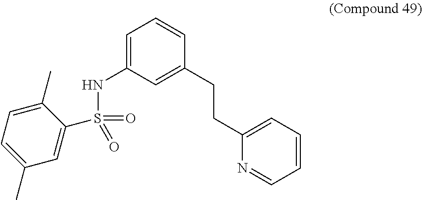

APCI-MS m/z: 367.1 [MH+].

LC rt 4.0 min. UV 254 nm

### EXAMPLE 40

### 3,4-Dimethyl-N-[3-(2-pyridin-2-ylethyl)phenyl]benzenesulfonamide

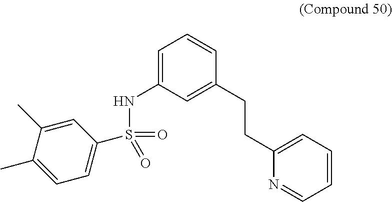

APCI-MS m/z: 367.1 [MH+].

LC rt 4.0 min. UV 254 nm

### EXAMPLE 41

### Human Glucocorticoid Receptor (GR) Assay

The assay is based on a commercial kit from Panvera/Invitrogen (Part number P2893). The assay technology is fluorescence polarization. The kit utilises recombinant human GR (Panvera, Part number P2812), a Fluoromone™ labelled tracer (GS Red, Panvera, Part number P2894) and a Stabilizing Peptide 10X (Panvera, Part number P2815). The GR and Stabilizing Peptide reagents are stored at −70° C. while the GS Red is stored at −20° C. Also included in the kit are 1M DTT (Panvera, Part number P2325, stored at −20° C.) and GR Screening buffer 10X (Panvera, Part number P2814, stored at −70° C. initially but once thawed stored at room temperature). Avoid repeated freeze/thaws for all reagents. The GR Screening buffer 10X comprises 100 mM potassium phosphate, 200 mM sodium molybdate, 1 mM EDTA and 20% DMSO.

Test compounds (1 μL) and controls (1 μL) in 100% DMSO were added to black polystyrene 384-well plates (Greiner low volume black flat-bottom, part number 784076). 0% control was 100% DMSO and 100% control was 10 μM Dexamethasone. Background solution (84; assay buffer 10X, Stabilizing Peptide, DTT and ice cold MQ water) was added to the background wells. GS Red solution (7 μL; assay buffer 10X, Stabilizing Peptide, DTT, GS Red and ice cold water) was added to all wells except background wells. GR solution (7 μL; assay buffer 10X, Stabilizing Peptide, DTT, GR and ice cold water) was added to all wells. The plate was sealed and incubated in a dark at room temperature for 2 hours. The plate was read in an Analyst plate reader (LJL Biosystems/Molecular Devices Corporation) or other similar plate reader capable of recording fluorescence polarization (excitation wavelength 530 nm, emission wavelength 590 nM and a dichroic mirror at 561 nm). The IC50 values were calculated using XLfit model 205.

| Compound nr | IC₅₀ (nM) |
|---|---|
| 1 | 14 |
| 2 | 35 |
| 5 | 370 |
| 6 | 700 |
| 19 | 36 |
| 20 | 59 |
| 21 | 62 |
| 22 | 99 |
| 23 | 110 |
| 24 | 170 |
| 25 | 170 |
| 26 | 200 |
| 27 | 200 |
| 28 | 220 |
| 29 | 230 |
| 30 | 310 |
| 31 | 330 |
| 32 | 390 |
| 33 | 650 |
| 34 | 660 |
| 35 | 710 |
| 36 | 810 |
| 37 | 820 |
| 49 | 350 |

1. A compound of formula (I): 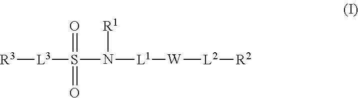
wherein:
R³ is phenyl optionally substituted by U, X, Y and Z; or thienyl, furyl or pyrazolyl all optionally substituted by X, Y and Z;
L³ is a bond or CH₂;
U, X, Y and Z are, independently, hydrogen, halo, C₁₋₆ alkyl, C₁₋₆ alkoxy, C₁₋₄ alkylthio, C₁₋₄ fluoroalkyl, C₁₋₄ fluoroalkoxy, phenyl (optionally substituted by halo, C₁₋₄ alkyl, C₁₋₄ fluoroalkyl, C₁₋₄ alkoxy or C₁₋₄ fluoroalkoxy), pyridyl (optionally substituted by halo, C₁₋₄ alkyl, C₁₋₄ fluoroalkyl, C₁₋₄ alkoxy or C₁₋₄ fluoroalkoxy), pyrazolyl (optionally substituted by halo, C₁₋₄ alkyl, C₁₋₄ fluoroalkyl, C₁₋₄ alkoxy or C₁₋₄ fluoroalkoxy), benzyloxy (optionally substituted by halo, C₁₋₄ alkyl, C₁₋₄ fluoroalkyl, C₁₋₄ alkoxy or C₁₋₄ fluoroalkoxy), phenoxy (optionally substituted by halo, C₁₋₄ alkyl, C₁₋₄ fluoroalkyl, C₁₋₄ alkoxy or C₁₋₄ fluoroalkoxy), pyridyloxy (optionally substituted by halo, C₁₋₄ alkyl, C₁₋₄ fluoroalkyl, C₁₋₄ alkoxy or C₁₋₄ fluoroalkoxy), nitro, cyano, S(O)₂NH₂, C(O)(C₁₋₄ alkyl), C(O)NH₂, NHC(O)(C₁₋₄ alkyl) or NR⁴R⁵; or X and Y join to form a fused benzene or pyridine ring;
R⁴ and R⁵ are, independently hydrogen, C₁₋₄ alkyl or C₃₋₇ cycloalkyl;
R¹ is hydrogen or C₁₋₄ alkyl;
W is a phenyl, isoxazolyl or pyrazolyl, cyclohexyl ring, or an acenaphthene ring system;
W being optionally substituted by halo or C₁₋₄ alkyl;
L¹ is a bond or CH₂;
L² is a bond, O, NH, (CH₂)ₙ or CH₂NH;
n is 1 or 2;
R² cyclohexyl, phenyl, methylenedioxyphenyl thienyl, pyrazolyl, thiazolyl, isoxazolyl, pyridinyl, pyrimidinyl, pyridazinyl, pyrazinyl, 1,3,5-triazinyl, 1,2,3-triazinyl, 1,2,4-triazinyl, benzofuranyl, benzthienyl, indolyl, indolinyl, dihydroindolinyl, indazolyl, benzimidazolyl, benzoxazolyl, benzthiazolyl, quinolinyl, tetrahydroquinolinyl, isoquinolinyl, quinoxalinyl, quinazolinyl, cinnolinyl, phthalazinyl, [1,8]-naphthiridinyl, [1,6]-naphthiridinyl, quinolin-2(1H)-onyl, isoquinolin-1(2H)-onyl, phthalazin-1(2H)-onyl, 1H-indazolyl, 1,3-dihydro-2H-indol-2-onyl, isoindolin-1-onyl, 3,4-dihydro-1H-isochromen-1-onyl, 1H-isochromen-1-onyl,
or an acenaphthene ring system;
R² is optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl), NHC(O)(C₁₋₄ alkyl) or NR⁶R⁷;
R⁶ and R⁷ are, independently, hydrogen, C₁₋₄ alkyl or C₃₋₇ cycloalkyl;
or a pharmaceutically acceptable salt thereof.

2. A compound as claimed in claim 1 wherein R¹ is hydrogen.

3. A compound as claimed in claim 1 wherein L¹ is CH₂.

4. A compound as claimed in claim 1, wherein L³ is a bond.

5. A compound as claimed in claim 1, wherein L² is CH₂CH₂.

6. A compound as claimed in claim 1 wherein W is phenyl.

7. A compound as claimed in claim 1 wherein R³ is phenyl (optionally substituted by halogen C₁₋₄ alkyl, C₁₋₄ haloalkyl, C₁₋₄ alkoxy or C₁₋₄ haloalkoxy), pyridyl (optionally substituted by halogen, C₁₋₄ alkyl, C₁₋₄ haloalkyl, C₁₋₄ alkoxy or C₁₋₄ haloalkoxy) or pyrazolyl (optionally substituted by C₁₋₄ alkyl, C₁₋₄ haloalkyl or phenyl (itself optionally substituted by halogen, C₁₋₄ alkyl, C₁₋₄ haloalkyl, C₁₋₄ alkoxy or C₁₋₄ haloalkoxy)).

8. A compound as claimed in claim 1 wherein R² is phenyl, methylenedioxyphenyl, pyridinyl, benzofuranyl, benzthienyl, indolyl, indolinyl, dihydroindolinyl, benzimidazolyl, benzoxazolyl, benzthiazolyl, quinolinyl, indazolyl, tetrahydroquinolinyl, isoquinolinyl, quinoxalinyl, quinazolinyl, cinnolinyl, phthalazinyl, [1,8]-naphthiridinyl or [1,6]-naphthiridinyl, optionally substituted as defined in claim 1.

9. A compound as claimed in claim 1 wherein R² is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃) or C(O)NH₂.

10. A process for the preparation of a compound of formula (I) as claimed in claim 1 comprising coupling a compound of formula (II): 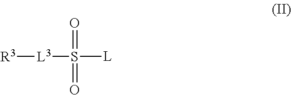
wherein L is a leaving group, with a compound of formula (III): 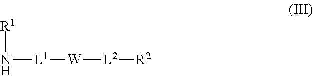
in a suitable solvent at a temperature in the range −10° C. to 50° C.

11. A pharmaceutical composition comprising a compound or formula (I) as claimed in claim 1, or a pharmaceutically acceptable salt thereof, and a pharmaceutically acceptable adjuvant, diluent or carrier.

12-13. (canceled)

14. A method of treating a glucocorticoid receptor mediated disease state in a mammal, which comprises administering to a mammal in need of such treatment an effective amount of a compound of formula (I), or a pharmaceutically acceptable salt thereof.
